(19) United States
(12) Patent Application Publication
(10) Pub. No.: US 2025/0149124 A1
(43) Pub. Date: May 8, 2025

(54) METHOD AND APPARATUS WITH NEURAL NETWORK TO MEASURE PROCESS-SEQUENCES SIMILARITIES AND TRAINING THEREOF

(71) Applicants: Samsung Electronics Co., Ltd., Suwon-si (KR); NEW YORK UNIVERSITY, New York, NY (US)

(72) Inventors: Seongmin OK, Suwon-si (KR); Kyunghyun CHO, New York, NY (US)

(73) Assignees: Samsung Electronics Co., Ltd., Suwon-si (KR); NEW YORK UNIVERSITY, New York, NY (US)

(21) Appl. No.: 18/890,610

(22) Filed: Sep. 19, 2024

Related U.S. Application Data
(60) Provisional application No. 63/547,777, filed on Nov. 8, 2023.

(30) Foreign Application Priority Data
Feb. 15, 2024 (KR) 10-2024-0022042

(51) Int. Cl. G16C 20/10; G16C 20/70

(52) U.S. Cl. CPC G16C 20/10; G16C 20/70

(57) ABSTRACT

A training method for similarity measurement and a measuring apparatus for similarity measurement and operating method thereof are provided. A method, performed by a computing device, includes: based on a first training parameter, embedding vectors of first processes included in a first process-sequence and embedding vectors of second processes included in a second process-sequence, respectively; based on a second training parameter, mapping first embedding vectors respectively corresponding to the first processes to second embedding vectors respectively corresponding to the second processes; and based on a result of the mapping, determining a similarity score indicating a similarity between the first process-sequence and the second process-sequence.

### CROSS-REFERENCE TO RELATED APPLICATIONS

This application claims the benefit under 35 USC § 119(e) of U.S. Provisional Application No. 63/547,777 filed on Nov. 8, 2023, in the U.S. Patent and Trademark Office, and claims the benefit under 35 USC § 119(a) of Korean Patent Application No. 10-2024-0022042 filed on Feb. 15, 2024, in the Korean Intellectual Property Office, the entire disclosures, all of which, are incorporated herein by reference for all purposes.

### BACKGROUND

### 1. Field

The following description relates to a training method for similarity measurement and a measuring apparatus for similarity measurement and operating method thereof.

### 2. Description of Related Art

In processes for the synthesis (e.g., creation) of an inorganic material, the final result is significantly influenced by, during the process, changes in temperature, environment, and the timing and amount of introduced substances over time. To achieve specific properties and yields, or to optimize synthesis, variations of a synthesis process may be explored by performing the process while adjusting various of the aforementioned variables.

For efficient exploration of a synthesis process, it may be helpful to analyze how the processes conducted in such variation experiments are related, what similarities the processes share, and also, the extent to which a new process to be explored differs from the processes previously experimented.

For data-based analysis of variations of a synthesis process, it is useful to measure the similarity between sequences of a single variable (e.g., temperature) without considering various processes or to measure the similarity between various processes without considering sequence aspects.

When optimizing processes, it is common to analyze selected variables at particular time points using methods such as regression analysis, rather than analyzing the entire sequence. In the typical process optimization, the results are obtained by comparing one timestamp with another timestamp.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method, performed by a computing device, includes: based on a first training parameter, embedding vectors of first processes included in a first process-sequence and embedding vectors of second processes included in a second process-sequence, respectively; based on a second training parameter, mapping first embedding vectors respectively corresponding to the first processes to second embedding vectors respectively corresponding to the second processes; and based on a result of the mapping, determining a similarity score indicating a similarity between the first process-sequence and the second process-sequence.

A process included in the first process-sequence and a process included in the second process-sequence may be independent of each other, and a number of the first processes and a number of the second processes may not be predetermined.

The first embedding vectors and the second embedding vectors may represent a process type or a process parameter, the process type includes a categorical variable, and the process parameter includes a continuous variable and a categorical variable.

The mapping of the first embedding vectors corresponding to the first processes to the second embedding vectors corresponding to the second processes is performed using dynamic programming.

The determining of the similarity score may be based on the first training parameter and the second training parameter that are pretrained with respect to a target property.

The determining of the similarity score may include: outputting a similarity matrix corresponding to similarity between the first process-sequence and the second process-sequence; and normalizing the similarity matrix by inputting the similarity matrix into a multivariate distribution model.

In another general aspect, a training method performed by a computing device includes: based on a first training parameter, embedding each of first processes included in a first process-sequence as respective first embedding vectors including a vector corresponding to a corresponding process type and at least one vector corresponding to at least one process parameter according to the corresponding process type; based on the first training parameter, embedding second processes included in a second process-sequence as respective second embedding vectors including a vector corresponding to a corresponding process type and at least one vector corresponding to at least one process parameter according to a corresponding process type; based on a second training parameter, mapping first embedding vectors respectively corresponding to the first processes to second embedding vectors respectively corresponding to the second processes; based on a result of the mapping, determining a similarity score indicating similarity between the first process-sequence and the second process-sequence; and based on a loss of a multivariate distribution model for the determined similarity score, training the first training parameter and the second training parameter.

The training of the first training parameter and the second training parameter may include training the first training parameter for embedding the first process as the first embedding vector and the second process as the second embedding vector.

The training of the first training parameter and the second training parameter may include training the second training parameter as a predefined matrix function for determining a vector similarity between the first process-sequence and the second process-sequence.

The multivariate distribution model may be a Gaussian processing model.

The training of the first training parameter and the second training parameter may include terminating training when the loss of the multivariate distribution model reaches a predetermined level of a numerical value preset for a target result.

The first embedding vectors may be mapped to the second embedding vectors using dynamic programming.

A non-transitory computer-readable storage medium may store instructions that, when executed by a processor, cause the processor to perform any of the methods.

In another general aspect, an apparatus includes: one or more processors; a memory storing instructions configured to cause the one or more processors to: based on a first training parameter, perform first embedding by embedding respective first processes included in a first process-sequence as a first embedding vector corresponding to a corresponding process type and at least one vector corresponding to at least one process parameter according to the corresponding process type; based on the first training parameter, perform second embedding by embedding second processes included in a second process-sequence as respective second embedding vectors corresponding to a corresponding process type and corresponding to at least one process parameter according to a corresponding process type; based on a second training parameter, map first embedding vectors corresponding to the first processes to respective second embedding vectors corresponding to the second processes; and based on a result of the mapping, determine a similarity score of similarity between the first process-sequence and the second process-sequence.

A process included in the first process-sequence and a process included in the second process-sequence may be independent of each other, and a number of the first processes and a number of the second processes may not be predetermined.

A process type may include a categorical variable, and a process parameter may include a continuous variable and a categorical variable.

The mapping of the first embedding vector to the second embedding vector may be performed using dynamic programming.

The similarity score may be determined based on the first training parameter and the second training parameter that are pretrained with respect to a target property.

The determining of the similarity score may include: outputting a similarity matrix of similarity between the first process-sequence and the second process-sequence; and normalizing the similarity matrix by inputting the similarity matrix into a multivariate distribution model.

Other features and aspects will be apparent from the following detailed description, the drawings, and the claims.

### BRIEF DESCRIPTION OF THE DRAWINGS

FIG. 1 illustrates an example of operation of a training apparatus that trains a method of measuring a similarity between process-sequences, according to one or more embodiments.

FIG. 2 illustrates an example of operating a training apparatus for training a method of measuring a similarity between process-sequences, according to one or more embodiments.

FIG. 3 illustrates an example of mapping between embedding vectors, according to one or more embodiments.

FIG. 4 illustrates an example of operating a measuring apparatus, according to one or more embodiments.

FIGS. 5A and 5B illustrate examples of using a measuring apparatus, according to one or more embodiments.

FIG. 6 illustrates an example of a measuring apparatus for measuring a similarity between process-sequences, according to one or more embodiments.

Throughout the drawings and the detailed description, unless otherwise described or provided, it may be understood that the same or like drawing reference numerals refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

The terminology used herein is for describing various examples only and is not to be used to limit the disclosure. The articles “a,” “an,” and “the” are intended to include the plural forms as well, unless the context clearly indicates otherwise. As used herein, the term “and/or” includes any one and any combination of any two or more of the associated listed items. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof.

Throughout the specification, when a component or element is described as being “connected to,” “coupled to,” or “joined to” another component or element, it may be directly “connected to,” “coupled to,” or “joined to” the other component or element, or there may reasonably be one or more other components or elements intervening therebetween. When a component or element is described as being “directly connected to,” “directly coupled to,” or “directly joined to” another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

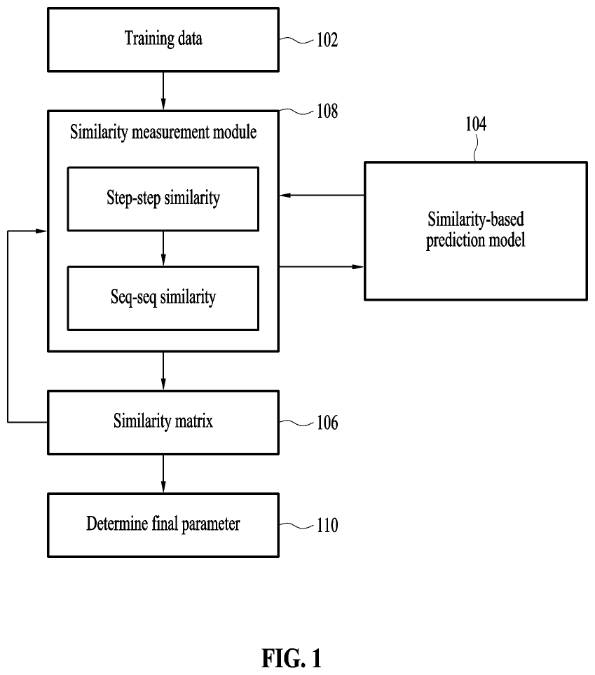

FIG. 1 illustrates an example of operation of a training apparatus that trains a method of measuring a similarity between process-sequences, according to one or more embodiments.

The training apparatus may train a similarity determination model included in a measuring apparatus. Particularly, parameters included in a similarity measurement model are trained. The similarity measurement model has a characteristic of a positive definite (PD) kernel.

Training data 102 may include a first process-sequence and a second process-sequence (“process-sequence” refers to a representation of a physical chemical/synthesis process). The number of processes (e.g., a temperature process or an addition process) included in the first process-sequence and the number of processes included in the second process-sequence may be the same or different. In addition, each of the processes may differ in type, extent, a number of steps/actions, and order. Generally, as used herein, a “process” is a discrete step or event or action in a synthesis procedure. The processes may also be referred to as steps or procedures. A “process-sequence” is a sequence of processes, each of which may have a timestamp.

Basically, default values of parameters for similarity measurement may be determined based on a similarity-based prediction model 104.

The parameters for similarity measurement may be broadly classified into two types. Firstly, there may be parameters for embedding vectors representing respective processes included in each sequence, that is, parameters for defining an embedding rule (step-step similarity in FIG. 1). Secondly, there may be parameters for mapping between points (here, may be a process) with high similarity based on a time domain between two sequences (seq-seq similarity in FIG. 1).

These two types of parameters may be yielded through a calculation to satisfy a property of the PD kernel. When the two types of parameters are determined and (based thereon) a similarity matrix 106 between the first process-sequence and the second process-sequence is output, an optimized result may be yielded for the similarity matrix 106 through a similarity-based model 104. The similarity-based model 104 may be, for example, a multivariate distribution model, for example, a Gaussian process model, a support vector machine (SVM), and k-nearest neighbors (k-NN). Hereinafter, an example of using a Gaussian process model is described.

Parameters of a similarity measurement module 108 may be updated such that a result yielded through the Gaussian process model is aligned with a target property. Based on a result of the updating, parameters of the similarity matrix 106 may be changed and a similarity between the first process-sequence and the second process-sequence may be measured again.

The Gaussian process model may be used to optimize parameters in a way that aligns with a target property associated with the PD kernel.

In this way, a parameter of a formula representing the similarity matrix and vector values of a vector representing a process may be updated and the finally determined parameter 110 may be used to represent a similarity matrix between two sequences input into the training apparatus for a corresponding target property.

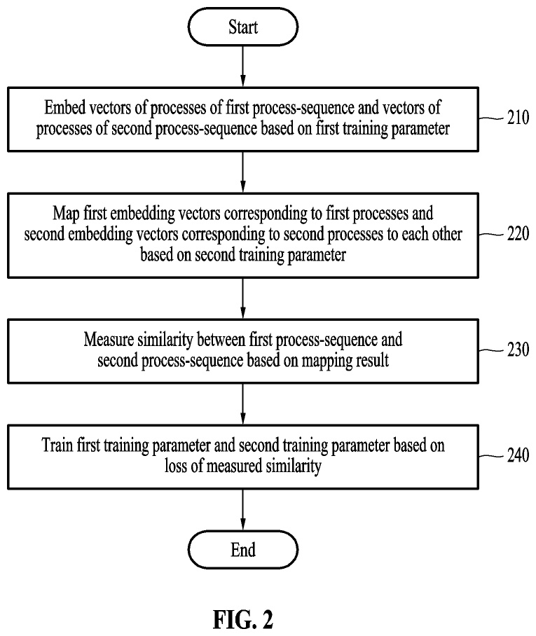

FIG. 2 illustrates an example of operating a training apparatus for training a method of measuring a similarity between two process-sequences, according to one or more embodiments.

Referring to FIG. 2, through operations 210 to 240, a training apparatus for measuring a similarity between two process-sequences (hereinafter, “training apparatus”) may train a training parameter.

When calculating a similarity between to process-sequences, the degree of the similarity indicates a determined/predicted similarity for a result of the process-sequences, e.g., an amount or yield from the process-sequences, a degree of a target property of the material produced by the process-sequences, or any other aspect of the result of the process-sequences.

First and second process-sequences may have sets of respective timestamps of the respective processes thereof. Each timestamp for an individual process within a process-sequence may be commonly associated with multiple variables for the timestamp (in other words, each process may have a timestamp and multiple variables that are parameters/features of the process, as described later). The variables of a process may be, for example, a set of mixed-type tokens combining continuous variables, such as temperature, input quantity, pressure, and categorical variables, such as input material and atmospheric conditions such as vacuum and nitrogen. There are no limitations on the number or types of variables that may constitute the processes.

In operation 210, the training apparatus may embed, based on a first training parameter, vectors of respective processes included in the first process-sequence and vectors of respective processes included in a second process-sequence.

The training apparatus may use the first training parameter (noted above) to embed each of first processes (included in the first process-sequence) as a respective first embedding vector including a (1-1)-th vector (corresponding to a corresponding process type) and at least one (1-2)-th vector corresponding to at least one process parameter according to a corresponding process type.

The training apparatus may use the same first training parameter (as used with the first process-sequence) to embed second processes (included in the second process-sequence). In this case, the training apparatus may use the first training parameter to embed each of the second processes as a respective second embedding vector including a (2-1)-th vector corresponding to a corresponding process type and at least one (2-2)-th vector corresponding to at least one process parameter according to a corresponding process type.

An example in which the training apparatus performs embedding using the first training parameter is described next.

For example, when a first process of a quantum dot (QD) synthesis process is a process of “adding 1.5 grams (g) of substance 1 at 20 degrees”, this first process may be represented by a vector in the form of (“adding a substance”, “substance 1”, 1.5, 20), and the like. Another process of “heating to 100 degrees”, as a second process, may be represented by (“heating”, “none”, N/A, 100).

In this example, a trainable first training parameter may be used to show similarities between the vectors representing the first process and the vectors representing th second process. Vectors representing respective processes may be represented as (or transformed to) numerical vectors (vectors whose elements are numbers) through the first training parameter.

Referring to the examples described above, for terms like “adding a substance”, represented by (add1, add2), “substance 1”, represented by (sub1, sub2), and “heating”, represented by (heat1, heat2), multiple real variables, such as add1, add2, sub1, sub2, heat1, and heat2 may be designated and randomly initialized.

Furthermore, according to a method described below, a final similarity may be expressed by a formula of parameters, and each parameter may be optimized in a way that effectively represents a target property between two process-sequences using this similarity as training data.

The training apparatus may represent a process as numerical vectors and determine a similarity between vectors in various ways. For example, a similarity between (x1, x2, x3) and (y1, y2, y3) may be determined to be e\hat{ }(−a|x1−y1|)*e\hat{ }(−b|x2−y2|)*e\hat{ }(−c|x3−y3|), and parameters indicating relative importance (for similarity) among first, second, and third elements are a, b, and c, which may be adjusted.

When a similarity is defined in with the formula above, a property of a PD kernel may be satisfied. Parameters to be trained according to a method described below may correspond to (or be) parameters of a PD kernel matrix function.

In operation 220, the training apparatus may map, based on a second training parameter, first embedding vectors corresponding to first processes to second embedding vectors corresponding to second processes.

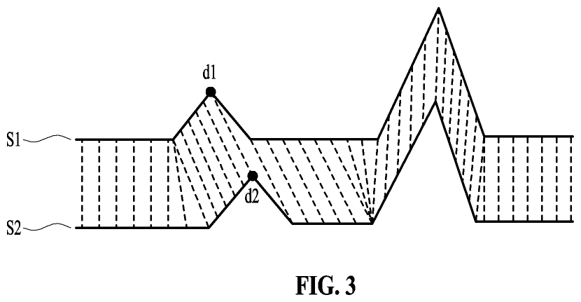

Mapping between embedding vectors may be performed by dynamic programming. Particularly, mapping may be performed based on a known matching technique called dynamic time warping (DTW). A method of mapping between embedding vectors is described with reference to FIG. 3.

FIG. 3 illustrates an example of mapping between embedding vectors, according to one or more embodiments. FIG. 3 shows two time series, S1 and S2 (explanatory examples which could be process-sequences). The horizontal direction in FIG. 3 represents time (from left to right), and the vertical direction in FIG. 3 represents a value.

In order to measure a similarity between the two time series S1 and S2, each point of S1 may be paired with each point of S2, and a distance between the points in each such paring may be determined (i.e., each pairing has a corresponding distance computed). For the result shown in FIG. 3, each point in S1 may be matched to the point in S2 that has the smallest distance among the points in S2. The process is referred to as dynamic time warping because a point in one time series may be mapped to a point in the other time series that is “closest” (in terms of values), even if they do not have the same time. For example, point d1 is time series S1 may be matched with and point d2 in time series S2, even though they have different times.

DTW may be used for measuring a similarity between time series. FIG. 3 shows an example where values of a time series are single scalar values. However, when the points included in S1 and S2 are represented by respective vectors (e.g., process vectors), mapping may be performed using a PD kernel when corresponding vectors are matched. Training data and a training parameter may satisfy a property of the PD kernel to perform machine learning.

A training apparatus may (i) express a similarity between processes using a formula based on a first parameter, as described earlier, in order to measure a similarity between the first process-sequence and the second process-sequence and may (ii) use a formula representing the similarity between the processes based on a second parameter to measure the similarity between the two sequences. In this case, a similarity matrix between the first process-sequence and the second process-sequence may be measured/determined.

In operation 230, the training apparatus may measure the similarity between the first process-sequence and the second process-sequence based on a mapping result.

The training apparatus may obtain the similarity matrix representing the similarity between the first process-sequence and the second process-sequence. For example, for two process-sequences each including 100 processes, a matrix value of 100×100 may be obtained (each element of the matrix being a pairing between a first process and a second process).

The training apparatus may obtain an optimization result by inputting the measured/determined similarity matrix into a multivariate distribution model. The multivariate distribution model may be a Gaussian process model, for example. A result value (a measured similarity) based on the similarity matrix (as optimized with the first training parameter and the second training parameter for each of the first process-sequence and the second process-sequence) may be obtained through the Gaussian process model.

In operation 240, the training apparatus may train, based on a loss of the measured similarity (as compared to a ground truth, for example), the first training parameter and the second training parameter.

Values of the first training parameter and the second training parameter may be updated such that a matrix function based on the first training parameter and the second training parameter is optimized for a target property.

For example, the first training parameter and the second training parameter may be updated in a recursive process that adjusts a parameter sequentially (through each recursion) to reach a predetermined value for a categorical parameter.

Parameters of a similarity measurement module (e.g., the similarity measurement module 108) may be updated such that a result yielded through the Gaussian process model is aligned with the target property. Based on a result of the updating, parameters of the similarity matrix may be changed and the similarity between the first process-sequence and the second process-sequence may be measured again. In this way, parameters of a formula expressing a similarity matrix and vector values representing a process may be updated.

Through an iterative process, the first training parameter and the second training parameter may be determined. Referring to the example described above, a, b, and c values in e\hat{ }(−a|x1−y1|)*e\hat{ }(−b|x2−y2|)*e\hat{ }(−c|x3−y3|) representing the similarity between (x1, x2, x3) and (y1, y2, y3) may be determined.

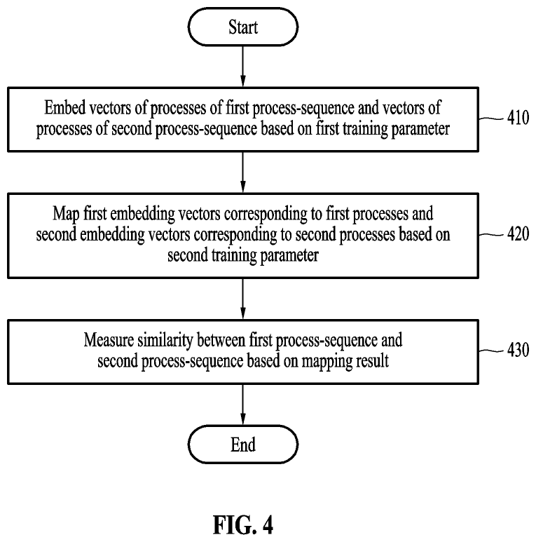

FIG. 4 illustrates an example of operating a measuring apparatus, according to one or more embodiments.

The measuring apparatus may measure a similarity between a first process-sequence and a second process-sequence based on parameters trained through a training apparatus (e.g., as described above). In describing FIG. 4, the terms “first process-sequence” and the “second process-sequence” do no not refer to the same process-sequences as the first process-sequence and the second process-sequence described above with reference to FIGS. 1 to 3. Furthermore, a first training parameter and a second training parameter of FIG. 4 correspond to determined training parameters, the training of which is completed as described with reference to FIGS. 1 to 3.

In operation 410, the measuring apparatus may embed, based on the first training parameter, vectors of processes of the first sequence and the second sequence.

The measuring apparatus may embed each of the first processes included in the first process-sequence respective first embedding vectors including a (1-1)-th vector corresponding to a corresponding process type and at least one (1-2)-th vector corresponding to at least one process parameter according to a corresponding process type.

The measuring apparatus may similarly embed second processes included in the second process-sequence using the first training parameter.

In operation 420, the measuring apparatus may map, based on the second training parameter, first embedding vectors respectively corresponding to the first processes to second embedding vectors respectively corresponding to the second processes.

Based on the second training parameter, mapping between sequences may be performed by a DTW-based mapping technique for processes represented by the vectors included in each of the first process-sequence and the second process-sequence.

As described above, mapping between sequences or embeddings of sequences may be performed based on the first training parameter and the second training parameter to satisfy a characteristic of a PD kernel during embedding and mapping phases.

In operation 430, the measuring apparatus may measure, based on a mapping result, the similarity between the first process-sequence and the second process-sequence.

A similarity matrix by the PD kernel may be measured based on a result of mapping between the sequences. The first training parameter and the second training parameter may be optimized through training and may be used to represent the similarity matrix representing the similarity between the two sequences in response to a target property.

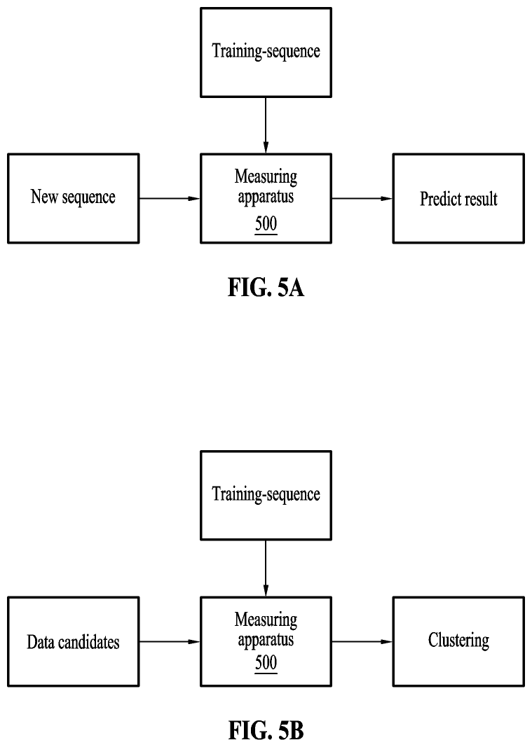

FIGS. 5A and 5B illustrate examples of using a measuring apparatus, according to one or more embodiments.

Referring to FIG. 5A, a measuring apparatus 500 may be used to predict a result for a new sequence.

When an existing process-sequence used for training and a newly generated process-sequence are input into the measuring apparatus 500, a process result for the newly generated process-sequence may be predicted using a similarity matrix yielded for the two sequences.

For example, it may be possible, for a new sequence, to predict the extent to which a result is yielded in response to a target property.

In addition, it may be possible to assess the extent of the difference between the new sequence and the sequence used in training. Therefore, based on a predicted process result when completing a process using the new sequence, it may be possible to determine whether to attempt the process or predict the likelihood of success.

Referring to FIG. 5B, the measuring apparatus 500 may be used for clustering sequences.

By inputting process-sequences, along with training-sequences, into the measuring apparatus 500 for data analysis for the process-sequences, a tendency of the process-sequences may be identified. For example, processes determined to yield a valid result related to the target property within a key process of a process-sequence may be clustered.

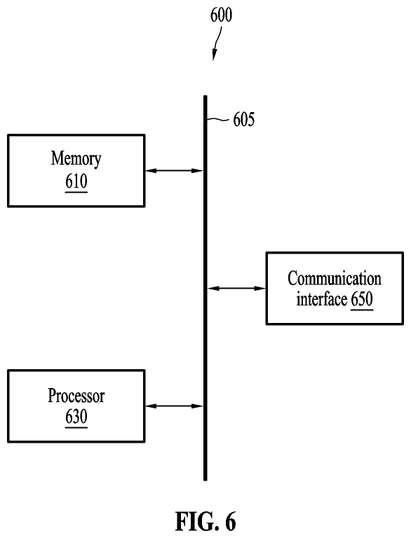

FIG. 6 illustrates an example of a measuring apparatus for measuring a similarity between process-sequences, according to one or more embodiments.

Referring to FIG. 6, a measuring apparatus 600 may include a communication interface 650, a processor 630, and a memory 610. The communication interface 650, the processor 630, and the memory 610 may communicate with each other via a communication bus 605.

The communication interface 650 may receive two process-sequences for similarity measurement.

The processor 630 may measure a similarity between the two process-sequences received through the communication interface 650. The processor 630 may measure the similarity using a matrix function expressed based on a pre-trained parameter through a training apparatus.

The memory 610 may store a variety of information generated by the processing process of the processor 630 described above. In addition, the memory 610 may store a variety of data and programs. The memory 610 may include a volatile memory or a non-volatile memory. The memory 610 may include a large-capacity storage medium such as a hard disk to store the variety of data.

Also, the processor 630 may perform one or more of the methods described with reference to FIGS. 1 to 5 or an algorithm corresponding to one or more of the methods. The processor 630 may be a data processing device implemented by hardware including a circuit having a physical structure to perform desired operations. The desired operations may include, for example, code or instructions included in a program. The processor 630 may be implemented as, for example, a central processing unit (CPU), a graphics processing unit (GPU), or a neural network processing unit (NPU). The measuring apparatus 600 that is implemented as hardware may include, for example, a microprocessor, a CPU, a processor core, a multi-core processor, a multiprocessor, an application-specific integrated circuit (ASIC), and a field programmable gate array (FPGA).

The processor 630 may execute a program and control the measuring apparatus 600. Program code to be executed by the processor 630 may be stored in the memory 610.

The computing apparatuses, the electronic devices, the processors, the memories, the displays, the information output system and hardware, the storage devices, and other apparatuses, devices, units, modules, and components described herein with respect to FIGS. 1-6 are implemented by or representative of hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-6 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-Res, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above disclosure, the scope of the disclosure may also be defined by the claims and their equivalents, and all variations within the scope of the claims and their equivalents are to be construed as being included in the disclosure.

What is claimed is:

1. A method, performed by a computing device, the method comprising:
based on a first training parameter, embedding vectors of first processes comprised in a first process-sequence and embedding vectors of second processes comprised in a second process-sequence, respectively;
based on a second training parameter, mapping first embedding vectors respectively corresponding to the first processes to second embedding vectors respectively corresponding to the second processes; and
based on a result of the mapping, determining a similarity score indicating a similarity between the first process-sequence and the second process-sequence.

2. The method of claim 1, wherein a process comprised in the first process-sequence and a process comprised in the second process-sequence are independent of each other, and a number of the first processes and a number of the second processes are not predetermined.

3. The method of claim 1, wherein
the first embedding vectors and the second embedding vectors represent a process type or a process parameter,
the process type comprises a categorical variable, and
the process parameter comprises a continuous variable and a categorical variable.

4. The method of claim 1, wherein the mapping of the first embedding vectors corresponding to the first processes to the second embedding vectors corresponding to the second processes is performed using dynamic programming.

5. The method of claim 1, wherein the determining of the similarity score is based on the first training parameter and the second training parameter that are pretrained with respect to a target property.

6. The method of claim 1, wherein the determining of the similarity score comprises:
outputting a similarity matrix corresponding to similarity between the first process-sequence and the second process-sequence; and
normalizing the similarity matrix by inputting the similarity matrix into a multivariate distribution model.

7. A training method performed by a computing device, the training method comprising:
based on a first training parameter, embedding each of first processes comprised in a first process-sequence as respective first embedding vectors comprising a vector corresponding to a corresponding process type and at least one vector corresponding to at least one process parameter according to the corresponding process type;
based on the first training parameter, embedding second processes comprised in a second process-sequence as respective second embedding vectors comprising a vector corresponding to a corresponding process type and at least one vector corresponding to at least one process parameter according to a corresponding process type;
based on a second training parameter, mapping first embedding vectors respectively corresponding to the first processes to second embedding vectors respectively corresponding to the second processes;
based on a result of the mapping, determining a similarity score indicating similarity between the first process-sequence and the second process-sequence; and
based on a loss of a multivariate distribution model for the determined similarity score, training the first training parameter and the second training parameter.

8. The training method of claim 7, wherein the training of the first training parameter and the second training parameter comprises training the first training parameter for embedding the first process as the first embedding vector and the second process as the second embedding vector.

9. The training method of claim 7, wherein the training of the first training parameter and the second training parameter comprises training the second training parameter as a predefined matrix function for determining a vector similarity between the first process-sequence and the second process-sequence.

10. The training method of claim 7, wherein the multivariate distribution model is a Gaussian processing model.

11. The training method of claim 7, wherein the training of the first training parameter and the second training parameter comprises terminating training when the loss of the multivariate distribution model reaches a predetermined level of a numerical value preset for a target result.

12. The training method of claim 7, wherein the first embedding vectors are mapped to the second embedding vectors using dynamic programming.

13. A non-transitory computer-readable storage medium storing instructions that, when executed by a processor, cause the processor to perform the training method of claim 7.

14. An apparatus comprising:
one or more processors;
a memory storing instructions configured to cause the one or more processors to:
based on a first training parameter, perform first embedding by embedding respective first processes comprised in a first process-sequence as a first embedding vector corresponding to a corresponding process type and at least one vector corresponding to at least one process parameter according to the corresponding process type;
based on the first training parameter, perform second embedding by embedding second processes comprised in a second process-sequence as respective second embedding vectors corresponding to a corresponding process type and corresponding to at least one process parameter according to a corresponding process type;
based on a second training parameter, map first embedding vectors corresponding to the first processes to respective second embedding vectors corresponding to the second processes; and
based on a result of the mapping, determine a similarity score of similarity between the first process-sequence and the second process-sequence.

15. The apparatus of claim 14, wherein a process comprised in the first process-sequence and a process comprised in the second process-sequence are independent of each other, and a number of the first processes and a number of the second processes are not predetermined.

16. The apparatus of claim 14, wherein
a process type comprises a categorical variable, and
a process parameter comprises a continuous variable and a categorical variable.

17. The apparatus of claim 14, wherein the mapping the first embedding vectors to the second embedding vectors is performed using dynamic programming.

18. The apparatus of claim 15, wherein the similarity score is determined based on the first training parameter and the second training parameter that are pretrained with respect to a target property.

19. The apparatus of claim 15, wherein the determining of the similarity score comprises:
outputting a similarity matrix of similarity between the first process-sequence and the second process-sequence; and
normalizing the similarity matrix by inputting the similarity matrix into a multivariate distribution model.
